(19) United States
(12) Patent Application Publication
(10) Pub. No.: US 2009/0318720 A1
(43) Pub. Date: Dec. 24, 2009

(54) Process for selectively synthesising 3 alpha-hydroxychlormadinone acetate

(75) Inventor: Thomas Otten, Roetgen (DE)

Correspondence Address: NORRIS, MCLAUGHLIN & MARCUS, PA, 875 THIRD AVENUE, 18TH FLOOR, NEW YORK, NY, 10022, US

(73) Assignee: GRUNENTHAL GMBH, Aachen (DE)

(21) Appl. No.: 12/468,191

(22) Filed: May 19, 2009

(30) Foreign Application Priority Data
May 21, 2008 (EP) 08 009 329.7

(51) Int. Cl. C07J 7/00

(52) U.S. Cl. 552/592

(57) ABSTRACT

The invention relates to a process for synthesising 3α-hydroxychlormadinone acetate.

The invention relates to a process for selectively synthesising 3α-hydroxychlormadinone acetate (I) and new intermediate products.

It is known that chlormadinone acetate can be used as an effective gestagen component for contraception or hormone replacement therapy. It is further known that chlormadinone acetate is metabolised, inter alia, by the metabolites 17α-acetoxy-6-chloro-3α-hydroxy-4,6-pregnandien-20-one (3α-hydroxychlormadinone acetate) and 17α-acetoxy-6-chloro-3β-hydroxy-4,6-pregnandien-20-one (3β-hydroxychlormadinone acetate) (see S. Honma et al., Chem. Pharm. Bull. 1977 (25) 2019-2031).

The aforementioned 3α- and 3β-hydroxy metabolites of chlormadinone acetate exhibit anti-androgenic and gestagen properties and can also be used for contraception and hormone replacement therapy (see WO 2007/085420, WO 2007/098828).

The processes known from the prior art for synthesising 3-hydroxy metabolites of chlormadinone acetate are not satisfactory, for example on account of the yield and regioselectivity or stereoselectivity thereof.

It is therefore an object of the invention to provide a process for preparing 3α- or 3β-hydroxychlormadinone acetate which provides advantages over the processes of the prior art.

This object is achieved by the subject-matter described hereinbelow.

It has unexpectedly been found that reducing chlormadinone acetate with an achiral hydride component to form 3β-hydroxychlormadinone acetate and subsequently inverting the configuration in position 3 enables a high yield of the desired 3α-hydroxychlormadinone acetate in a highly pure form to be obtained.

The invention relates to a process for preparing 3α-hydroxychlormadinone acetate (I)

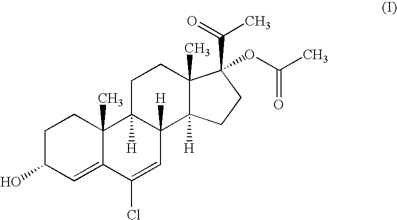

comprising the steps of
a) reducing chlormadinone acetate (II)

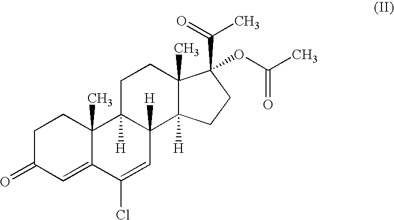

with a hydride component to form 3β-hydroxychlormadinone acetate (III)

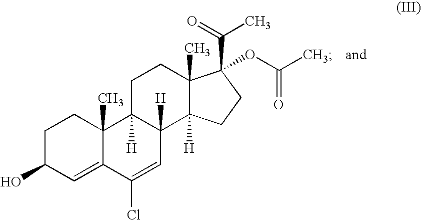

b) inverting the configuration of 3β-hydroxychlormadinone acetate (III) in position 3 to form 3α-hydroxychlormadinone acetate (I).

The substance amount ratio (mol/mol) of the hydride component to chlormadinone acetate (II) in step a) is preferably at least 1.38, more preferably at least 1.5, even more preferably at least 2.0, most preferably at least 2.5 and in particular at least 3.0. In a particularly preferred embodiment, the substance amount ratio is at least 3.5.

In a preferred embodiment of the process according to the invention, the chlormadinone acetate concentration in step a) is between 0.0010 and 1.0 mol/l, more preferably between 0.0020 and 0.75 mol/l, even more preferably between 0.0050 and 0.50 mol/l, most preferably between 0.0080 and 0.25 and in particular between 0.020 and 0.060 mol/l. In a particularly preferred embodiment the chlormadinone acetate concentration in step a) is 0.040±0.010 mol/l.

In a further preferred embodiment, the hydride component concentration in step a) is between 0.010 and 10 mol/l, more preferably between 0.025 and 5.0 mol/l, even more preferably between 0.050 and 1.0 mol/l, most preferably between 0.075 and 0.50 mol/l and in particular between 0.10 and 0.20 mol/l. In a particularly preferred embodiment the hydride component concentration in step a) is 0.14±0.05 mol/l.

The hydride component used in step a) as a reducing agent is preferably achiral, i.e. it is optically inactive, and therefore does not have a stereogenic element, i.e. does not have a stereogenic centre, axis or plane.

The terms “optical activity”, “stereogenic element”, “stereogenic centre”, stereogenic axis” and “stereogenic plane” are known to the person skilled in the art and are defined for example in G. P. Moss, Basic terminology of stereochemistry, Pure & Applied Chemistry 1996 (68) 2193-2222. The term “asymmetric” is also frequently used in the prior art in place of the term “stereogenic”.

The term “hydride component” is preferably to be understood in the meaning of the description as a chemical compound capable of transferring hydride anions (H⁻) (hydride donor). For example, acetone (CH₃)₂CO is reduced in the presence of a hydride component to form i-propanol (CH₃)₂CHOH, during which process a hydride anion is transferred from the hydride component (hydride donor) to the carbonyl carbon atom of the acetone (hydride acceptor). This is officially described as the addition of the hydride anion to the carbonyl carbon atom, which leads to the reduction of acetone to i-propanol.

The hydride component of the process according to the invention is preferably a metal hydride preferably containing at least one metal selected from the group consisting of lithium, sodium, potassium, magnesium, calcium, boron, aluminium, silicon, tin and zinc.

The aforementioned metal hydride more preferably contains at least one metal selected from the group consisting of lithium, sodium, potassium, boron and aluminium, even more preferably at least one metal selected from the group consisting of lithium, sodium and boron and most preferably at least one metal selected from the group consisting of sodium and boron. In a particularly preferred embodiment, the metal is sodium.

In a further preferred embodiment of the process according to the invention, the metal hydride is selected from the group consisting of LiH, NaH, MgH₂, MeOMgH, MeOMg₂H₃, CaH₂, BH₃, (Me)₂CHC(Me)₂BH₂, (CF₃COO)₂BH, LiBH₄, NaBH₄, KBH₄, Ca(BH₄)₂, (Me₄N)BH₄, (Et₄N)BH₄, (n-Bu₄N)BH₄, LiH₃BMe, LiH₃B(n-Bu), LiH₃BCH₂CN, LiH₃BC(Me)₂CN, NaHB(OMe)₃, NaHB(O-i-Pr)₃, NaHB(O-t-Bu)₃, KHB(O-i-Pr)₃, NaHB(OAc)₃, KHB(OAc)₃, (Me₄N)HB(OAc)₃, (n-Bu₄N)HB(OAc)₃, LiHBEt₃, KHBEt₃, LiHB(sec-Bu)₃, KHB(sec-Bu)₃, KHBPh₃, A1H₃, i-Bu₂AlH, LiAlH₄, NaAlH₄, LiHAl(OCH₃)₃, LiHAl(O-t-Bu)₃, LiHAl(OCEt₃)₃, Et₃SiH, PhMe₂SiH, PH₃SiH, Et₂SiH₂, Ph₂SiH₂, PhSiH₃, (MeO)₃SiH, (EtO)₃SiH, n-Bu₂SnH₂, n-Bu₂SnFH, n-Bu₂SnClH, Ph₂SnH₂, n-Bu₃SnH, Ph₃SnH, Zn(BH₄)₂, LiBH₃N(i-Pr)₂, NaBH₃NMe₂, LiBH₃CN, NaBH₃CN and (Et₄N)BH₃CN.

Furthermore, the following metal hydrides may preferably also be used:

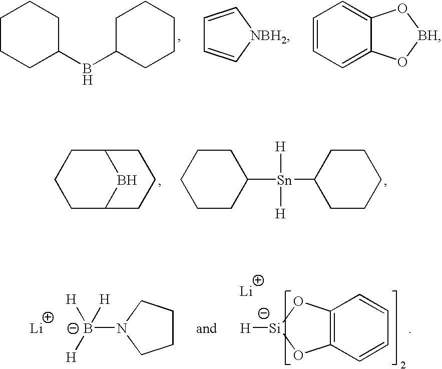

Moreover, the metal hydride is
more preferably selected from the group consisting of LiH, NaH, BH₃, LiBH₄, NaBH₄, KBH₄, (Me₄N)BH₄, (Et₄N)BH₄, (n-BU₄N)BH₄, NaHB(OMe)₃, NaHB(O-i-Pr)₃, NaHB(O-t-Bu)₃, KHB(O-i-Pr)₃, NaHB(OAc)₃, KHB(OAc)₃, (Me₄N)HB(OAc)₃, (n-BU₄N)HB(OAc)₃, AlH₃, i-Bu₂AlH, LiAlH₄, NaAlH₄, LiBH₃CN, NaBH₃CN and (Et₄N)BH₃CN,
even more preferably selected from the group consisting of NaH, BH₃, LiBH₄, NaBH₄, NaHB(OMe)₃, NaHB(OAc)₃, i-Bu₂AlH, LiAlH₄, NaAlH₄ and NaBH₃CN,
most preferably selected from the group consisting of BH₃, LiBH₄, NaBH₄, NaAlH₄ and NaBH₃CN.

The metal hydride is particularly preferably NaBH₄.

In cases in which BH₃ (borane) is used as a hydride component, the BH₃, which is itself a Lewis acid (electron pair acceptor), may preferably be complexed with Lewis bases (electron pair donors). Complexes of this type are generally also known as Lewis adducts and examples thereof include borane-1,2-bis(t-butylthio)ethane complex, borane-4-methylmorpholine complex, borane-NH₃ complex, borane-THF complex, borane-di(t-butyl)phosphine complex, borane-dimethylsulphide complex, borane-dimethylamine complex, borane-diphenylphosphine complex, borane-isoamylsulphide complex, borane-morpholine complex, borane-N,N-diethylaniline complex, borane-N,N-diisopropylethylamine complex, borane-pyridine complex, borane-t-butylamine complex, borane-triethylamine complex, borane-trimethylamine complex, borane-triphenylphosphine complex, borane-2-picoline complex and borane-t-butyldimethylphosphine complex.

Additives which can increase the reaction rate for example may also preferably be added to the reduction reaction in step a). Suitable additives include, for example, Ti(O-i-Pr)₄, TiCl₄, Et₃B, SnCl₄, Al₂O₃, Et₂BOMe, n-Bu₃B, CaCl₂, Cp₂TiCl₂ (Cp=cyclopentadiene), MnCl₂, NiCl₂, Me₃SiCl, PdCl₂, ZnCl₂, ZnBr₂, SmCl₃, CeCl₃, TiCl₄, CeCl₃, MgBr₂, ZnCl₂, ZnBr₂, MgBr₂, LiI and AlCl₃.

In step a), solvents which are inert to the hydride component or react therewith relatively slowly may preferably be used in step a), i.e. the solvents themselves are not subject to reduction or are subject thereto to a limited extent only. Appropriate solvents are known to the person skilled in the art. Examples include water, alcohols (for example methanol, ethanol, n-propanol, i-propanol, n-butanol, t-butanol), ethers (for example diethyl ether, tetrahydrofuran), chlorinated hydrocarbons (for example chloroform, dichloromethane) and aromatic hydrocarbons (for example xylene, toluene, benzene) or mixtures of the aforementioned solvents.

The reaction components may preferably be added in any desired order. In a particularly preferred embodiment, chlormadinone acetate is placed in a reaction vessel and dissolved in one of the aforementioned solvents. The solution can subsequently be brought to the desired temperature. The hydride component is then preferably added slowly and in portions to the reaction solution.

The temperature is preferably varied as a function of the reactivity of the hydride component used. In general, the more reactive the hydride component, the lower the reaction temperature should be in order, inter alia, to carry out a controlled reaction, reduce the formation of by-products and avoid laboratory accidents on account of the heat produced by the reduction reaction. Therefore the reaction temperature may be between −48° C. and +100° C. for example, depending on the hydride component selected.

Highly reactive hydride components, such as LiAlH₄, i-Bu₂AlH and NaAlH₄ are preferably reacted at between −48° C. and +20° C., more preferably between −48° C. and 0° C. Reactive hydride components, such as LiBH₄, NaBH₄, NaHB(OMe)₃, NaHB(OAc)₃ and NaBH₃CN are preferably reacted at between −20° C. and +30° C., more preferably between −10° C. and +15° C., and even more preferably between −5.0° C. and +5.0° C. Less reactive hydride components, such as BH₃ mentioned above and the Lewis adducts thereof are preferably reacted at between −5.0° C. and +100° C., more preferably between +5.0° C. and +50° C., and even more preferably between +10° C. and +35° C.

Methods for raising and lowering the reaction temperature are known to the person skilled in the art. For example, a reaction mixture may be cooled down to −48° C. by a mixture of acetone and liquid nitrogen used as an external cooling means. Reducing the temperature to 0° C. can be achieved by using ice as an external cooling means for example.

The person skilled in the art is aware that the reaction rate varies greatly as a function of the reactivity and concentration of the reaction partners, the solvent, the temperature, etc. and the reaction time may thus also vary. In general, the higher the reactivity of the reaction partners or the higher the temperature of the reaction mixture, the higher the reaction rate and the shorter the reaction time.

Reaction time is preferably to be understood in the meaning of the description as the time interval from the moment the reaction partners are brought into contact (t₀) to the moment the starting materials are no longer reacting to form products (tₓ). In an ideal case, tₓ is preferably the moment at which at least one of the starting materials has been completely consumed and further reaction is therefore not possible. The reaction time is preferably calculated as follows: reaction time=tₓ−t₀. Methods for determining the reaction rate and time are known to the person skilled in the art. For example, the progress of a reaction can be tracked by HPLC, LC-MS, GC-MS and/or thin layer chromatography.

When using highly-reactive hydride components, such as LiAlH₄, i-Bu₂AlH and NaAlH₄, the reaction time is generally preferably between 1 second and 5.0 hours in length, more preferably between 1 minute and 2.5 hours, even more preferably between 2.5 and 60 minutes, most preferably between 5.0 and 30 minutes and in particular between 10 and 20 minutes. If reactive hydride components, such as LiBH₄, NaBH₄, NaHB(OMe)₃, NaHB(OAc)₃ and NaBH₃CN are used, the reaction time is typically preferably between 1 minute and 10 hours, more preferably between 10 minutes and 7.5 hours, even more preferably between 20 minutes and 5.0 hours, most preferably between 30 minutes and 3.0 hours and in particular between 1.0 and 2.0 hours. The use of less reactive hydride components, such as BH₃ mentioned above and the Lewis adducts thereof, generally results in a longer reaction time, this reaction time preferably being between 30 minutes and 7.0 days, more preferably between 1 hour and 3.0 days, even more preferably between 2.0 and 24 hours, most preferably between 3.0 and 16 hours and in particular between 4.0 and 12 hours.

Upon completion of the reaction, i.e. preferably once tₓ has been reached, the excess hydride component is preferably destroyed by adding water, organic or inorganic acids (ammonium chloride, hydrochloric acid, acetic acid for example) or organic or inorganic oxidants (acetone, sodium hypochlorite for example). The excess hydride component (for example NaBH₄) is preferably destroyed by adding acetone to the reaction mixture.

The reaction mixture is worked up using methods which are known to the person skilled in the art. After the excess hydride component has been destroyed, water is preferably added to the reaction mixture and the mixture obtained is subsequently preferably extracted using a solvent which is immiscible with water. Organic solvents such as ethyl acetate, chloroform, dichloromethane, diethyl ether, hexane and pentane may be used for the extraction process.

After extraction is complete, a desiccant (for example sodium sulphate, magnesium sulphate) is preferably added to the separated organic phase to bind or separate the water present in the organic phase.

The dried organic phase may then preferably be filtered to separate off the desiccant. The filtered organic phase may subsequently preferably be evaporated to a low volume. Methods for evaporating solvents are known to the person skilled in the art. The organic phase may preferably be evaporated in a rotary evaporator, optionally at reduced pressure and/or optionally at elevated temperature. The residue obtained after the organic phase has been evaporated may subsequently preferably be purified further. Purification methods are known to the person skilled in the art. Purification may be carried out for example using flash chromatography, preferably on silica gel, preparative HPLC or recrystallisation.

It has surprisingly been found that the reduction of chlormadinone acetate in step a) in accordance with the process according to the invention results in a high diastereomeric excess of the desired 3β-hydroxychlormadinone acetate (III).

In this way, the diastereomeric excess (d.e.) of 3β-hydroxychlormadinone acetate is preferably at least 75% d.e., more preferably at least 80% d.e., even more preferably at least 85% d.e., most preferably at least 90% d.e. and in particular at least 94% d.e.

Methods for determining the diastereomeric excess are known to the person skilled in the art. For example, suitable methods include, inter alia, ¹H NMR and HPLC.

It is particularly surprising that the high diastereomeric excess of 3β-hydroxychlormadinone acetate is obtained through the use of an achiral, optically inactive reduction agent such as NaBH₄.

It is especially surprising given that such high stereoselectivity can generally only be obtained in asymmetric synthesis by using chiral reducing agents, such as B-isopinocampheyl-9-borabicyclo[3.3.1]nonane, or chiral catalysts, such as the Corey-Bakshi-Shibata catalyst. These aforementioned chiral reducing agents or chiral catalysts cause the reaction partners to have a defined spatial orientation in relation to one another and consequently preferentially cause a stereoisomer to be formed (asymmetric synthesis). Chiral reducing agents and chiral catalysts have the drawback that they are very expensive in comparison with achiral reducing agents. The use of achiral hydride components, such as NaBH₄, in the process according to the invention therefore enables cost savings to be made.

Furthermore, reactions between chlormadinone acetate and NaBH₄ are known from the prior art, but provide a relatively low yield (approx. 50%) of 3β-hydroxychlormadinone acetate and a 12% yield of twice-reduced diol as a by-product (see J. Fiet et al., Steroids 2002 (67) 1045-1055).

It is also extremely surprising that a high yield of 3β-hydroxychlormadinone acetate can be obtained by carrying out the reaction in a suitable manner. For example, the yield of 3β-hydroxychlormadinone acetate in step a) is preferably at least 60%, more preferably at least 70%, even more preferably at least 80%, most preferably at least 90% and in particular at least 98% relative to the amount of the starting material used (chlormadinone acetate).

It has been found that, when carrying out the reaction in step a), the yield of 3β-hydroxychlormadinone acetate can be positively influenced in particular by the reaction temperature, the absolute concentration of chlormadinone acetate or the hydride component and the relative stoichiometric ratio of the hydride component to the chlormadinone acetate.

This high yield is all the more surprising in view of the prior art, since the process according to the invention results in regioselective reduction of the carbonyl function in position 3, but the carbonyl function of the acetyl group in position 17 is not reduced, or is not reduced to any significant extent. In this way, only negligible amounts of by-products are formed in step a) of the process according to the invention.

The high stereoselectivity and regioselectivity of the reaction therefore provides the advantage that costly purification steps are not necessary to obtain 3β-hydroxychlormadinone acetate with a sufficient level of purity. The high level of purity enables the 3β-hydroxychlormadinone acetate to be used in the further synthesis steps without requiring prior purification. The process according to the invention is therefore a time-saving and cost-effective preparation process.

In step b) of the process according to the invention, position 3 is inverted.

In a preferred embodiment, step b) comprises the following steps:
b1) reacting 3β-hydroxychlormadinone acetate (III) with at least one carboxylic acid in the presence of
   b1″) at least one phosphine component and at least one azo component, or
   b1″) at least one phosphorane component
to form an ester of general formula (IV)

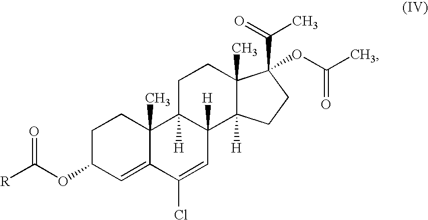

in which R is an unsubstituted or a mono- or polysubstituted C₁-C₁₂ hydrocarbon; and
b2) cleaving the ester (IV) to form 3α-hydroxychlormadinone acetate (I).

The term “inversion” in the meaning of the description preferably means a reversal of the configuration at a stereogenic centre. Inversion, in this case of 3β-hydroxychlormadinone acetate, preferably means that the configuration of the hydroxyl group in position 3 of 3βhydroxychlormadinone acetate is reversed from the β configuration into the α configuration.

A person skilled in the art would recognise that the reaction in step b) is, in the broadest sense, a Mitsunobu reaction or a variant thereof.

The aforementioned carboxylic acid is preferably defined by the general formula R—COOH, in which the residue R of the carboxylic acid and the ester (IV) represents a —C₁-C₁₂ hydrocarbon.

In the meaning of the description, a —C₁-C₁₂ hydrocarbon is preferably to be understood as a residue which contains carbon and hydrogen atoms and 1, 2, 3, 4, 5, 6, 7, 8, 9, 10, 11 or 12 carbon atoms. This residue may be unsubstituted or mono- or polysubstituted with the same or different residues selected from the group consisting of halogen, —CN, —NO, —NO₂, —C₁-C₆ alkyl, —C₁-C₆ perhalogenalkyl, —OH, —O—C₁-C₆ alkyl, —SH, —S—C₁-C₆ alkyl, —NH₂, —N(C₁-C₆ alkyl)₂, —NHC(═O)—C₁-C₆ alkyl, —NH(C═O)O—C₁-C₆ alkyl, —C(═O)—C₁-C₆ alkyl, —C(═O)O—C₁-C₆ alkyl, —C(═O)NH—C₁-C₆ alkyl and —SO₂—C₁-C₆ alkyl.

The term “hydrocarbon” is known to the person skilled in the art and is defined for example in G. P. Moss, Glossary of class names of organic compounds and reactive intermediates based on structure, Pure & Applied Chemistry 1995 (67) 1307-1375.

The hydrocarbon is preferably aliphatic or aromatic or contains both an aliphatic and an aromatic component. The hydrocarbon is preferably saturated or mono- or polyunsaturated. If the hydrocarbon is aliphatic, it may be acyclic and/or cyclic (alicyclic). If the hydrocarbon is acyclic, it may be linear or branched. The aliphatic hydrocarbons, i.e. the linear or branched acyclic hydrocarbons and the cyclic (alicyclic) hydrocarbons, may be saturated or mono- or polyunsaturated respectively.

Therefore, an aliphatic hydrocarbon is preferably to be understood in the meaning of the description as an acyclic or cyclic (alicyclic), saturated or mono- or polyunsaturated hydrocarbon residue which is not aromatic. Furthermore, an acyclic aliphatic hydrocarbon may preferably be unbranched (linear) or branched. If the aliphatic hydrocarbon is unsaturated, it may preferably have at least one double bond and/or at least one triple bond, preferably 1, 2 or 3 double bonds and/or triple bonds. Suitable saturated or unsaturated aliphatic C₁-C₁₂ hydrocarbons are for example methyl, ethyl, n-propyl, i-propyl, n-butyl, i-butyl, sec-butyl, t-butyl, n-pentyl, neo-pentyl, n-hexyl, n-heptyl, n-octyl, n-nonyl, n-decyl, n-undecyl, n-dodecyl, vinyl, allyl, ethynyl, cyclopropyl, cyclobutyl, cyclopentyl, cyclohexyl, cycloheptyl, cyclopentenyl and cyclohexenyl.

An unsaturated hydrocarbon may have one or more conjugated or non-conjugated C═C double or C≡C triple bonds (for example —CH═CH—CH═CH₂, —C≡C—C≡CH), or have both one or more C═C double bonds and one or more C≡C triple bonds simultaneously (for example —CH═CH—CH₂—C≡CH), which may in turn be conjugated or non-conjugated).

The terms “aliphatic” and “alicyclic” are known to the person skilled in the art and are defined for example in G. P. Moss, Glossary of class names of organic compounds and reactive intermediates based on structure, Pure & Applied Chemistry 1995 (67) 1307-1375.

Saturated, linear or branched, acyclic aliphatic hydrocarbons are conventionally also referred to as “alkyls” (for example methyl, ethyl, propyl, butyl). Correspondingly, unsaturated, linear or branched, acyclic aliphatic hydrocarbons with at least one C═C double bond are conventionally also referred to as “alkenyls” (for example ethenyl, propenyl, vinyl, allyl) and unsaturated, linear or branched, acyclic aliphatic hydrocarbons with at least one C≡C triple bond are conventionally also referred to as “alkynyls” (for example ethynyl).

Saturated, cyclic aliphatic hydrocarbons are conventionally also referred to as “cycloalkyls” (for example cyclopropyl, cyclobutyl, cyclopentyl, cyclohexyl). Unsaturated, cyclic, aliphatic hydrocarbons with at least one C═C double bond are conventionally referred to as “cycloalkenyls” (for example cyclopentenyl, cyclohexenyl).

The terms “alkyl”, “alkenyl”, “alkynyl”, “cycloalkyl” and “cycloalkenyl” are known to the person skilled in the art and are defined for example in G. P. Moss, Glossary of class names of organic compounds and reactive intermediates based on structure, Pure & Applied Chemistry 1995 (67) 1307-1375.

In the meaning of the description, the aliphatic hydrocarbon may preferably be formed from both an alicyclic component and an acyclic component, which may be linear or branched respectively. Examples include the following residues: cyclopentylmethyl, cyclohexylethyl, methylcyclopentyl and ethylcyclohexyl.

Aromatic hydrocarbons are known to the person skilled in the art. The aromatic hydrocarbon may be unfused (not annelated) or fused (annelated). The terms “aromatic” and “annelated” are known to the person skilled in the art and are defined for example in P. Muller, Glossary of terms used in physical organic chemistry, Pure & Applied Chemistry 1994 (66) 1077-1184. A suitable unfused (not annelated) aromatic hydrocarbon is phenyl for example. An example of a fused (annelated) aromatic hydrocarbon is naphthyl.

An aliphatic and aromatic hydrocarbon is preferably to be understood in the meaning of the description as a residue which contains both an aliphatic component and an aromatic component, the terms aliphatic and aromatic hydrocarbon being defined as described above. Suitable residues which contain both an aliphatic and an aromatic hydrocarbon include for example benzyl, methylphenyl, dimethylphenyl, mesityl, phenethyl, ethylphenyl, phenylpropyl, propylphenyl, naphthylmethyl, methylnaphthyl, naphthylethyl and ethylnaphthyl.

In a preferred embodiment, the residue R is preferably selected from the group consisting of —C₁-C₆ alkyl, —C₂-C₆ alkenyl, —C₂-C₆ alkynyl, —C₃-C₁₂ cycloalkyl, —C₁-C₆-alkyl-C₃-C₁₂ cycloalkyl, -aryl and —C₁-C₆-alkyl-aryl; in which the aforementioned residues may be unsubstituted or mono- or polysubstituted with the same or different residues selected from the group consisting of halogen, —CN, —NO, —NO₂, —C₁-C₆ alkyl, —C₁-C₆ perhalogenalkyl, —OH, —O—C₁-C₆ alkyl, —SH, —S—C₁-C₆ alkyl, —NH₂, —N(C₁-C₆ alkyl)₂, —NHC(═O)—C₁-C₆ alkyl, —NH(C═O)O—C₁-C₆ alkyl, —C(═O)—C₁-C₆ alkyl, —C(═O)O—C₁-C₆ alkyl, —C(═O)NH—C₁-C₆ alkyl and —SO₂—C₁-C₆ alkyl.

The term “halogen” is preferably to be understood in the meaning of the description as a residue selected from the group consisting of —F, —Cl, —Br and —I.

In the meaning of the description, the term “—C₁-C₆ perhalogenalkyl” is preferably to be understood to mean that all the hydrogen atoms of a —C₁-C₆ alkyl residue are replaced (substituted) by the same or different, preferably the same, halogen atoms. Examples include the following residues: —CF₃, —CCl₃ and —CF₂CF₃.

In a further preferred embodiment, the residue R is preferably selected from the group consisting of methyl, ethyl, n-propyl, i-propyl, n-butyl, i-butyl, sec-butyl, t-butyl, n-pentyl, neo-pentyl, n-hexyl, vinyl, allyl, ethynyl, cyclopropyl, cyclobutyl, cyclopentyl, cyclohexyl, phenyl, benzyl, phenethyl and naphthyl, in which the aforementioned residues may be unsubstituted or mono- or polysubstituted with the same or different substituents selected from the group consisting of —F, —Cl, —Br, —I, —CN, —NO₂, —CH₃, —CF₃, —OH, —OCH₃, —SH, —SCH₃, —NH₂, —N(CH₃)₂, —C(═O)CH₃, —C(═O)O—CH₃ and —SO₂CH₃.

In a particularly preferred embodiment, R is a phenyl, benzyl or naphthyl, in which the aforementioned residues are unsubstituted or mono- or polysubstituted with the same or different substituents selected from the group consisting of —F, —Cl, —Br, —CN, —NO₂, —CH₃, —CF₃, —OCH₃, —SCH₃, —N(CH₃)₂, —C(═O)CH₃, —C(═O)O—CH₃ and —SO₂CH₃.

R particularly preferably represents 4-cyanophenyl, 4-fluorophenyl, 4-chlorophenyl, 4-nitrophenyl, 4-(trifluoromethyl)phenyl, 4-(methylsulphonyl)phenyl, 4-acetylphenyl, 4-cyanonaphthyl, 4-fluoronaphthyl, 4-chloronaphthyl, 4-nitronaphthyl, 4-(trifluoromethyl)naphthyl, 4-(methylsulphonyl)naphthyl or 4-acetylnaphthyl.

The pKₛ value of the aforementioned carboxylic acid is preferably between 0.25 and 6.9, more preferably between 0.50 and 6.5, even more preferably between 1.0 and 6.0, most preferably between 1.5 and 5.5 and in particular between 2.0 and 5.0.

In a preferred embodiment, the phosphine component is a compound of general formula (V)

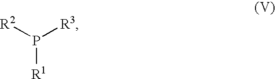

in which
R¹, R² and R³, are each selected, independently of one another, from the group consisting of C₁-C₁₂ alkyl, C₃-C₁₂ cycloalkyl, aryl and heteroaryl, in which the aforementioned residues may be unsubstituted or mono- or polysubstituted with the same or different residues selected from the group consisting of halogen, —CN, —NO, —NO₂, —C₁-C₆ alkyl, —C₁-C₆ perhalogenalkyl, —O—C₁-C₆ alkyl, —S—C₁-C₆ alkyl, —N(C₁-C₆ alkyl)₂, pyrrolidinyl, piperidinyl, N-methylpiperazinyl, morpholinyl, —NHC(═O)—C₁-C₆ alkyl, —NH(C═O)O—C₁-C₆ alkyl, —C(═O)—C₁-C₆ alkyl, —C(═O)O—C₁-C₆ alkyl and —C(═O)NH—C₁-C₆ alkyl.

In the meaning of the description, the term “heteroaryl” preferably represents a cyclic, aromatic hydrocarbon which preferably comprises 5, 6, 7, 8, 9, 10, 11 or 12 ring members and contains, as ring members and in addition to carbon atoms, one or more heteroatoms which are the same or different and are preferably selected from the group consisting of N, O and S. Suitable heteroaryls include pyridyl(pyridinyl), furyl(furanyl) and thienyl(thiophenyl) for example.

In a further preferred embodiment, the residues R¹, R² and R³ are each preferably selected, independently of one another, from the group consisting of methyl, ethyl, n-propyl, i-propyl, n-butyl, i-butyl, sec-butyl, t-butyl, n-pentyl, neo-pentyl, n-hexyl, n-heptyl, n-octyl, n-nonyl, n-decyl, n-undecyl, n-dodecyl, vinyl, allyl, cyclopropyl, cyclobutyl, cyclopentyl, cyclohexyl, cycloheptyl, phenyl, naphthyl, pyridyl, furyl and thienyl, in which the aforementioned residues may be unsubstituted or mono- or polysubstituted with the same or different substituents selected from the group consisting of —F, —Cl, —Br, —I, —CN, —NO₂, —CH₃, —CF₃, —OCH₃, —SCH₃, —N(CH₃)₂, pyrrolidinyl, piperidinyl, N-methylpiperazinyl, morpholinyl, —NHC(═O)—CH₃, —NH(C═O)O—CH₃, —C(═O)—CH₃, —C(═O)O—CH₃, —C(═O)O—CH₂CH₃, —C(═O)O—(CH₂)₂CH₃, —C(═O)O—CH(CH₃)₂, —C(═O)O—(CH₂)₃CH₃, —C(═O)O—C(CH₃)₃ and —C(═O)NH—CH₃.

In a particularly preferred embodiment, the phosphine component is selected from the group consisting of 4-(dimethylamino)phenyldiphenylphosphine, dicyclohexylphenylphosphine, diethylphenylphosphine, diphenyl-2-pyridylphosphine, isopropyldiphenylphosphine, tri-n-octylphosphine, tri-t-butylphosphine, tri-n-butylphosphine, tricyclohexylphosphine, tri-n-hexylphosphine, triphenylphosphine, tris[3,5-bis(trifluoromethyl)phenyl]phosphine, b is {4-[2-(perfluorocyclohexyl)ethyl]phenyl}(phenyl)phosphine and (3,3,4,4,5,5,6,6,7,7,8,8,9,9,10,10,10-heptadecafluorodecyl)diphenylphosphine.

Diphenylphosphino polystyrene resin and/or 4-diphenylphosphinomethyl polystyrene resin, which may preferably each be cross-linked with divinylbenzene, may also preferably be used as phosphine components.

The azo component is preferably a compound of general formula (VI)

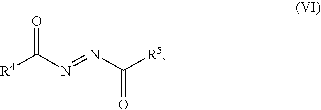

in which
R⁴ and R⁵ are each selected, independently of one another, from the group consisting of C₁-C₁₂ alkyl, —N(C₁-C₆ alkyl), pyrrolidinyl, piperidinyl, N-methylpiperazinyl, morpholinyl and aryl, in which the aforementioned residues may be unsubstituted or mono- or polysubstituted with the same or different residues selected from the group consisting of halogen, —CN, —NO₂, —C₁-C₆ alkyl, —C₁-C₆ perhalogenalkyl, —O—C₁-C₆ alkyl, —S—C₁-C₆ alkyl and —N(C₁-C₆ alkyl)₂. or
R₄ and R₅ together form the group

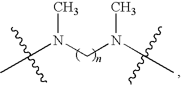

in which n represents 1, 2, 3 or 4.

In the context of the present description, the symbol

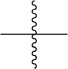

used in the above formula represents the link between the above group and the general formula (VI).

In a further preferred embodiment R⁴ and R⁵ are each selected, independently of one another, from the group consisting of methyl, ethyl, n-propyl, i-propyl, n-butyl, i-butyl, sec-butyl, t-butyl, n-pentyl, neo-pentyl, n-hexyl, N,N-dimethylamino, N,N-diethylamino, N,N-di-n-propylamino, N,N-di-1-propylamino, N,N-di-n-butylamino, N,N-di-1-butylamino, N,N-di-sec-butylamino, N,N-di-t-butylamino, pyrrolidinyl, piperidinyl, N-methylpiperazinyl, morpholinyl, phenyl and naphthyl, in which the aforementioned residues may be unsubstituted or mono- or polysubstituted with the same or different residues selected from the group consisting of F, Cl, Br, I, —O—CH₃, —O—CH₂CH₃, —O—(CH₂)₂CH₃, —O—CH(CH₃)₂, —O—(CH₂)₃CH₃, —O—C(CH₃)₃ and —S—CH₃; or

R₄ and R₅ together form the group

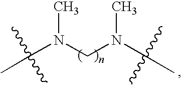

in which n represents 1, 2, or 3, preferably 2.

In a particularly preferred embodiment, the azo component is selected from the group consisting of 1,1′-(azodicarbonyl)dipiperidine, bis(2,2,2-trichlorethyl)azodicarboxylate, di-(4-chlorbenzyl)azodicarboxylate, di-t-butylazodicarboxylate, diethylazodicarboxylate, di-1-propylazodicarboxylate, di-(2-methoxyethyl)azodicarboxylate, (E)-N¹,N¹,N²,N²-tetramethyldiazene-1,2-dicarboxamide, (E)-N¹,N¹,N²,N²-tetraisopropyldiazene-1,2-dicarboxamide and (Z)-4,7-dimethyl-4,5,6,7-tetrahydro-1,2,4,7-tetrazocine-3,8-dione.

The phosphorane component is preferably a compound of general formula (VII)

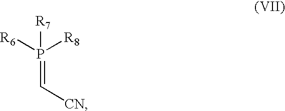

in which
R⁶, R⁷ and R⁸ each represent, independently of one another, a —C₁-C₁₂ hydrocarbon.

In a further preferred embodiment, R⁶, R⁷ and R⁸ each represent, independently of one another, a —C₁-C₁₂ alkyl, —C₃-C₁₂ cycloalkyl, —C₁-C₆-alkyl-C₃-C₁₂ cycloalkyl, aryl or —C₁-C₆-alkyl-aryl.

The residues R⁶, R⁷ and R⁸ are further preferably selected, independently of one another, from the group consisting of methyl, ethyl, n-propyl, i-propyl, n-butyl, i-butyl, sec-butyl, t-butyl, n-pentyl, neo-pentyl, n-hexyl, n-heptyl, n-octyl, n-nonyl, n-decyl, n-undecyl, n-dodecyl, cyclopropyl, cyclobutyl, cyclopentyl, cyclohexyl, cycloheptyl, phenyl, benzyl, phenethyl, phenylpropyl, naphthyl and naphthylmethyl.

In a particularly preferred embodiment, the phosphorane component is (cyanomethylene)tributylphosphorane or (cyanomethylene)trimethylphosphorane.

In a preferred embodiment, the substance amount ratio (mol/mol)
of carboxylic acid to 3β-hydroxychlormadinone acetate in step b1′) is at least 1.00, more preferably at least 1.10 and in particular 1.14±0.10;
of the phosphine component to 3β-hydroxychlormadinone acetate in step b1′) is at least 1.00, more preferably at least 1.04 and in particular 1.08±0.10; and/or
of the azo component to 3β-hydroxychlormadinone acetate in step b1′) is at least 1.00, more preferably at least 1.02 and in particular 1.05±0.10.

Preferably, the substance amount ratio (mol/mol)
of carboxylic acid to 3β-hydroxychlormadinone acetate in step b1″) is 1.00±0.10, more preferably 1.10±0.10 and in particular 1.14±0.10; and/or
of the phosphorane component to 3β-hydroxychlormadinone acetate in step b1″) is at least 1.00±0.10, more preferably at least 1.04±0.10 and in particular 1.10±0.10.

The concentrations of 3β-hydroxychlormadinone acetate, carboxylic acid, the phosphine component, azo component and phosphorane component in step b1) are, independently of one another, preferably between 0.0010 mol/l and 10 mol/l, more preferably between 0.0050 and 7.5 mol/l or 0.0075 and 5.0 mol/l, even more preferably between 0.010 and 2.5 mol/l or 0.025 and 1.0 mol/l, most preferably between 0.050 and 0.75 mol/l or 0.075 and 0.50 mol/l and in particular between 0.10 and 0.17 mol/l or 0.13 and 0.16 mol/l respectively.

In a preferred embodiment of the process according to the invention, non-polar aprotic and/or polar aprotic solvents may preferably be used, each independently of one another, in steps b1′) and b1″). Examples of non-polar aprotic solvents include benzene, toluene, xylene, n-hexane, n-pentane, cyclohexane, carbon tetrachloride and mixtures of the aforementioned solvents. Ethers, (for example diethyl ether, tetrahydrofuran), ketones (for example acetone), esters (for example ethyl acetate), asymmetrically chlorinated hydrocarbons (for example 1,1,1-trichloroethane) and mixtures of the aforementioned solvents may preferably be used as polar aprotic solvents. Non-polar aromatic solvents are particularly preferred.

The terms “polar aprotic” and “non-polar aprotic” and known to the person skilled in the art.

It has surprisingly been found that the diastereomer ratio can be improved by using non-polar aromatic solvents (for example benzene, toluene, xylene or mesitylene) instead of comparatively polar, non-aromatic solvents (for example, THF, CH₂Cl₂).

In a particularly preferred embodiment, benzene is used as a solvent.

The aforementioned solvents are preferably dried by suitable methods before use and are thus anhydrous.

The term “anhydrous solvent” is to be understood, in the meaning of the description, as a solvent preferably containing a maximum of 0.010%, more preferably a maximum of 0.0080%, even more preferably a maximum of 0.0060%, most preferably a maximum of 0.0040% and in particular a maximum of 0.0020% of water relative to the total weight of the solvent.

Methods for drying organic solvents are known to the person skilled in the art. For example, ethers, such as tetrahydrofuran or diethyl ether, may be distilled over sodium benzophenone ketyl under an inert gas atmosphere. Chlorinated solvents are also conventionally dried by distillation over calcium hydride (CaH₂) under an inert gas atmosphere.

Methods for determining the residual water content in dried solvents are known to the person skilled in the art. For example, the water content can be determined by Karl Fischer titration.

The temperatures of the reaction mixture in steps b1′) and b1″), independently of one another, are preferably between ±0.0° C. and +50° C., more preferably between +5.0° C. and +40° C., even more preferably between +10° C. and +35° C., most preferably between +15° C. and +30° C. and in particular between +20° C. and +25° C. (ambient temperature).

It has surprisingly been found that the diastereomer ratio deteriorates as the reaction temperature is reduced, and that optimal results can be obtained at room temperature.

Steps b1′) and b1″) are preferably carried out under anhydrous reaction conditions and/or under a protective gas atmosphere.

A person skilled in the art is aware of what is meant by the term “anhydrous reaction conditions”.

The reaction components may preferably be added in any desired order in steps b1′) and b1″).

In a particularly preferred embodiment, 3β-hydroxychlormadinone acetate and the phosphine component are added to anhydrous benzene. The carboxylic acid and the azo component are subsequently added in succession.

The azo component, after having previously been diluted by a dry solvent, preferably dry/anhydrous benzene, is preferably added dropwise over a time period of preferably at least one minute, more preferably at least 5.0 minutes, even more preferably at least 10 minutes, most preferably at least 15 minutes and in particular at least 20 minutes.

The reaction times of steps b1′) and b1″), independently of one another, are preferably between 0.50 and 96 hours, more preferably between 1.0 and 48 hours, even more preferably between 2.0 and 36 hours, most preferably between 4.0 and 24 hours and in particular between 12 and 20 hours. In a particularly preferred embodiment, the reaction time is 18±3.0 hours.

The reaction mixture is preferably worked up using methods which are known to the person skilled in the art.

In a particularly preferred embodiment, the reaction mixture is evaporated in a rotary evaporator, preferably at reduced pressure and/or elevated temperature (for example 40° C.), upon completion of the reaction. The resulting residue is preferably added to a solvent, such as diethyl ether, chloroform or dichloromethane, preferably dichloromethane. The resulting solution is preferably initially washed with an aqueous, alkaline solution, such as aqueous sodium carbonate, sodium bicarbonate or sodium hydroxide solution and subsequently washed with water and/or brine (saturated aqueous NaCl solution). The organic phase is then preferably dried over a desiccant, such as sodium sulphate or magnesium sulphate. The desiccant is then preferably filtered off and the filtered solution is preferably evaporated in a rotary evaporator. The resulting residue may subsequently preferably be purified by flash chromatography on silica gel, preparative HPLC or recrystallisation.

The yield of ester (IV) is preferably at least 55%, more preferably at least 60%, even more preferably at least 65%, most preferably at least 70% and in particular at least 74% relative to the amount of starting material used (3β-hydroxychlormadinone acetate).

The diastereomeric excess (d.e.) of the ester (IV) is preferably at least 50% d.e., more preferably at least 60% d.e., even more preferably at least 70% d.e., most preferably at least 80% d.e. and in particular at least 90% d.e. In a particularly preferred embodiment, the diastereomeric excess is 93±2% d.e.

It is known that only low Mitsunobu product yields can be obtained when reacting steroid compounds having double bonds in Ring A and/or in Ring B of the steroid skeleton. In addition, stereoselectivity may be poor when carrying out the inversion process with unsaturated steroid compounds of this type, resulting in a mixture of stereoisomers which requires further purification.

In this respect, reference is made for example to O. Mitsunobu, The use of diethyl azodicarboxylate and triphenylphosphine in synthesis and transformation of natural products, Synthesis 1981, 1-28. This document shows that reacting cholesterol with benzoic acid, diethyl azodicarboxylate and triphenylphosphine produces a complex mixture, largely composed of undesirable by-products. In addition, this complex mixture contains only a low yield (11%) of the corresponding inverted cholesterol ester, but a higher yield (20%) of the undesirable non-inverted cholesterol ester.

It has surprisingly been found that diunsaturated 3β-hydroxychlormadinone acetate can be reacted with a high degree of stereoselectivity to produce a high yield of the inverted ester (IV). The process according to the invention therefore avoids having to carry out a complicated product purification process which involves separating off the undesirable stereoisomers thereof and the by-products obtained.

The ester (IV) is preferably cleaved in step b2) in acid or alkaline reaction conditions.

Examples of organic and inorganic acids which may be used for the ester cleavage process include formic acid, acetic acid, propionic acid, hydrochloric acid and hydrobromic acid.

In a particularly preferred embodiment, the ester cleavage process is carried out in alkaline conditions. In this process, bases such as sodium carbonate, sodium bicarbonate, potassium carbonate, potassium bicarbonate, sodium hydroxide and/or potassium hydroxide are preferably used. In a particularly preferred embodiment, aqueous sodium hydroxide (sodium hydroxide solution) is used for the ester cleavage process.

Suitable preferable solvents for the ester cleavage process include water, alcohols (for example methanol, ethanol, n-propanol, i-propanol), ethers (for example THF) and mixtures of these solvents.

The concentrations of the ester (IV) and the base in the reaction mixture are, independently of one another, preferably between 0.0010 and 10 mol/l, more preferably between 0.0050 and 1.0 mol/l, even more preferably between 0.0075 and 0.50 mol/l, most preferably between 0.010 and 0.10 mol/l and in particular between 0.025 and 0.075 mol/l. In a particularly preferred embodiment, the concentration in the reaction mixture is 0.050±0.010 mol/l.

The substance amount ratio (mol/mol) of base to ester (IV) is preferably between 1.00 and 1.50, more preferably between 1.00 and 1.40, even more preferably between 1.00 and 1.30, most preferably between 1.00 and 1.20 and in particular between 1.00 and 1.10. In a particularly preferred embodiment, the substance amount ratio is 1.030±0.010.

The reaction time for the ester cleavage process is preferably between 5.0 and 120 minutes, more preferably between 10 and 100 minutes, even more preferably between 20 and 75 minutes, most preferably between 30 and 60 minutes and in particular between 40 and 50 minutes. In a particularly preferred embodiment, the ester cleavage reaction time is 45±3.0 minutes.

After the reaction is complete, the reaction mixture is preferably worked up using methods which are known to the person skilled in the art.

In a particularly preferred embodiment, the alkaline reaction solution is brought to a pH of preferably 7.0 to 7.5 (neutralisation) by an acid, preferably aqueous hydrochloric acid, at the beginning of the work-up stage. The neutralised solution is then preferably evaporated in a rotary evaporator. Water is subsequently preferably added to the residue and the resulting mixture is preferably extracted with an organic solvent, such as ethyl acetate, chloroform or dichloromethane. The organic phase is then preferably dried over a desiccant, such as sodium sulphate and magnesium sulphate. The organic phase is then preferably filtered and the filtrate may be purified. Purification methods are known to the person skilled in the art. For example, the residue may undergo flash chromatography on silica gel, preparative HPLC or a recrystallisation step.

The yield of 3α-hydroxychlormadinone acetate is preferably at least 50%, more preferably at least 55%, even more preferably at least 60%, most preferably at least 65% and in particular at least 69% relative to the amount of ester (IV) used.

The diastereomeric excess (d.e.) of 3α-hydroxychlormadinone acetate after the ester cleavage process b2) is preferably at least 50% d.e., more preferably at least 60% d.e., even more preferably at least 70% d.e., most preferably at least 75% d.e. and in particular at least 85% d.e. In a particularly preferred embodiment, the diastereomeric excess is at least 90±5% d.e.

Methods for determining the diastereomeric excess are known to the person skilled in the art. It may be determined for example by ¹H NMR or HPLC.

The 3β-hydroxychlormadinone acetate, which is present in very low amounts, may, if necessary, preferably be separated off by preparative HPLC over a suitable stationary phase (for example Gemini 5μ C18 110A).

The total 3α-hydroxychlormadinone acetate yield over the three reaction steps a), b1) and b2) is preferably 51±5.0%.

The invention also relates to compounds of general formula (IV)

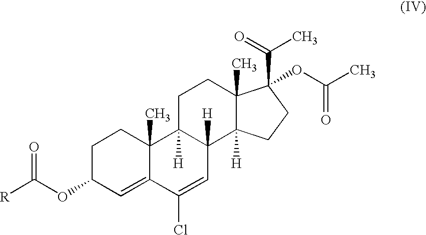

in which R is an unsubstituted or a mono- or polysubstituted C₁-C₁₂ hydrocarbon, provided that R is not ethyl, and is preferably neither methyl nor ethyl.

In a preferred embodiment, the residue R is preferably selected from the group consisting of methyl, —C₃-C₆ alkyl, —C₂-C₆ alkenyl, —C₂-C₆ alkynyl, —C₃-C₁₂ cycloalkyl, —C₁-C₆-alkyl-C₃-C₁₂ cycloalkyl, aryl and —C₁-C₆-alkyl-aryl; in which the aforementioned residues may be unsubstituted or mono- or polysubstituted with the same or different residues selected from the group consisting of halogen, —CN, —NO, —NO₂, —C₁-C₆ alkyl, —C₁-C₆ perhalogenalkyl, —OH, —O—C₁-C₆ alkyl, —SH, —S—C₁-C₆ alkyl, —NH₂, —N(C₁-C₆ alkyl)₂, —NHC(═O)—C₁-C₆ alkyl, —NH(C═O)O—C₁-C₆ alkyl, —C(═O)—C₁-C₆ alkyl, —C(═O)O—C₁-C₆ alkyl, —C(═O)NH—C₁-C₆ alkyl and —SO₂—C₁-C₆ alkyl.

In a further preferred embodiment, the residue R is preferably selected from the group consisting of methyl, n-propyl, i-propyl, n-butyl, i-butyl, sec-butyl, t-butyl, n-pentyl, neo-pentyl, n-hexyl, vinyl, allyl, ethynyl, cyclopropyl, cyclobutyl, cyclopentyl, cyclohexyl, phenyl, benzyl, phenethyl and naphthyl, in which the aforementioned residues may be unsubstituted or mono- or polysubstituted with the same or different substituents selected from the group consisting of —F, —Cl, —Br, —I, —CN, —NO₂, —CH₃, —CF₃, —OH, —OCH₃, —SH, —SCH₃, —NH₂, —N(CH₃)₂, —C(═O)CH₃, —C(═O)O—CH₃ and —SO₂—CH₃.

In a particularly preferred embodiment, R is a phenyl, benzyl or naphthyl, in which the aforementioned residues may be unsubstituted or mono- or polysubstituted with the same or different substituents selected from the group consisting of —F, —Cl, —Br, —CN, —NO₂, —CH₃, —CF₃, —OCH₃, —SCH₃, —N(CH₃)₂, —C(═O)CH₃, —C(═O)O—CH₃ and —SO₂—CH₃.

R particularly preferably represents 4-cyanophenyl, 4-fluorophenyl, 4-chlorophenyl, 4-nitrophenyl, 4-(trifluoromethyl)phenyl, 4-(methylsulphonyl)phenyl, 4-acetylphenyl, 4-cyanonaphthyl, 4-fluoronaphthyl, 4-chloronaphthyl, 4-nitronaphthyl, 4-(trifluoromethyl)naphthyl, 4-(methylsulphonyl)naphthyl or 4-acetylnaphthyl.

The compounds according to the invention of general formula (IV) are particularly suitable as intermediate compounds in the process of synthesising 3α-hydroxychlormadinone acetate (I) from chlormadinone acetate.

The following non-limiting examples are given only for the purposes of clarifying the invention.

### EXAMPLE 1

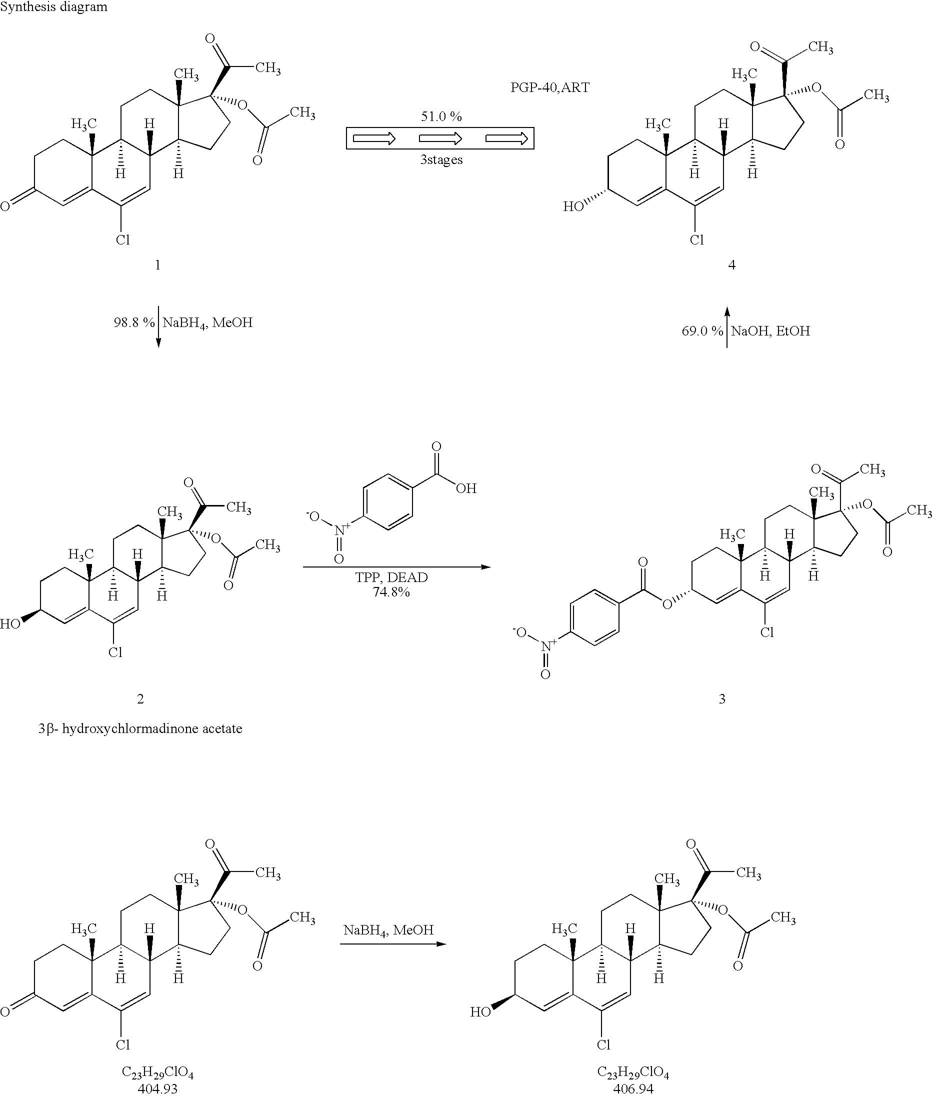

Chlormadinone acetate (5.0 g; 12.3 mmol) was suspended in methanol (307 ml) and was cooled to 0-5° C. by an ice bath. Sodium borohydride (1.63 g; 43.2 mmol) was added and the batch was stirred for 105 minutes. Acetone (15 ml) was subsequently added and the mixture was stirred for a further 10 minutes. The entire batch was poured into water (770 ml) and extracted four times with dichloromethane. The combined organic phases were dried over sodium sulphate, filtered off from the desiccant and evaporated. The residue was purified using flash chromatography (silica gel; chloroform/dichloromethane/diethyl ether: 5/5/1).

| | |
|---|---|
| Yield: | 4.98 g (98.8%), light yellow solid |
| Melting point: | 186.2° C. |
| α/β ratio: | 3.0/97.0 (¹H-NMR) |
| | 2.2/97.8 (HPLC) |
| Amount of rotation: | [α]_{D} = −93.38 (c = 1.0; MeOH) |
| LC-MS: | ^{m}/_{z} = 406.9 (MH⁺) |

¹H-NMR (400 MHz, CDCl₃): δ=0.69 (s, 3H); 1.03 (s, 3H); 1.14-1.22 (m, 1H); 1.27-1.48 (m, 3H); 1.52-1.99 (m, 10H); 2.05 (s, 3H); 2.08 (s, 3H); 2.24 (m, 1H); 2.96 (m, 1H); 4.33 (m, 1H); 5.87 (s, 1H); 6.08 (s, 1H) ppm.

¹³C-NMR (100 MHz, CDCl₃): δ=14.34; 18.14; 20.24; 21.17; 23.30; 26.43; 28.47; 30.30; 31.11; 33.79; 36.95; 38.28; 47.64; 49.01; 50.35; 68.00; 96.41; 128.22; 129.11; 130.78; 141.14; 170.66; 203.96 ppm.

### 3α-(4-nitrophenylcarboxy)chlormadinone acetate

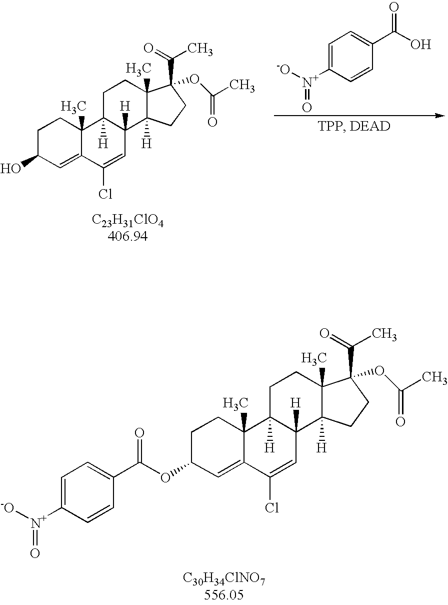

3β-hydroxychlormadinone acetate (3.05 g; 7.5 mmol) and triphenylphosphine (2.13 g; 8.1 mmol) were placed under a nitrogen atmosphere in dry benzene (55 ml) and were stirred for 10 minutes. 4-nitrobenzoic acid (1.44; 8.6 mmol) was subsequently added and the mixture was stirred for a further 15 minutes. Diethyl azodicarboxylate (3.7 ml; 7.9 mmol) as a 40% solution in toluene was diluted with dry benzene (11 ml) and was added slowly. The batch was stirred at ambient temperature for 18 hours. It was then evaporated and the residue was added to dichloromethane (250 ml) and washed with sodium carbonate solution and water. After it was dried over sodium sulphate, it was filtered off from the desiccant and evaporated. The residue was purified by flash chromatography (silica gel; chloroform/dichloromethane/diethyl ether: 5/5/1).

| | |
|---|---|
| Yield: | 3.12 g (74.8%), light yellow solid |
| Melting point: | 123.7° C. |
| α/β ratio: | 96.5/3.5 (¹H-NMR) |
| Amount of rotation: | [α]_{D} = +152.7 (c = 1.0; MeOH) |

¹H-NMR (400 MHz, CDCl₃): δ=0.72 (s, 3H); 1.02 (s, 3H); 1.28-2.14 (m, 13H); 2.06 (s, 3H); 2.09 (s, 3H); 2.31 (m, 1H); 2.99 (m, 1H); 5.66 (m, 1H); 6.01 (d, J=Hz, 1H); 6.27 (d, J=Hz, 1H); 8.21 (d, J=8.5 Hz, 2H); 8.28 (d, J=9.0 Hz, 2H) ppm.

¹³C-NMR (100 MHz, CDCl₃): δ=14.33; 16.77; 20.51; 21.19; 23.25; 24.46; 28.45; 30.31; 30.37; 31.10; 37.06; 38.16; 47.45; 49.00; 50.03; 68.11; 96.34; 120.31; 123.47; 130.69; 130.78; 131.12; 136.05; 145.66; 150.50; 164.11; 170.60; 203.84 ppm.

### 3α-hydroxychlormadinone acetate

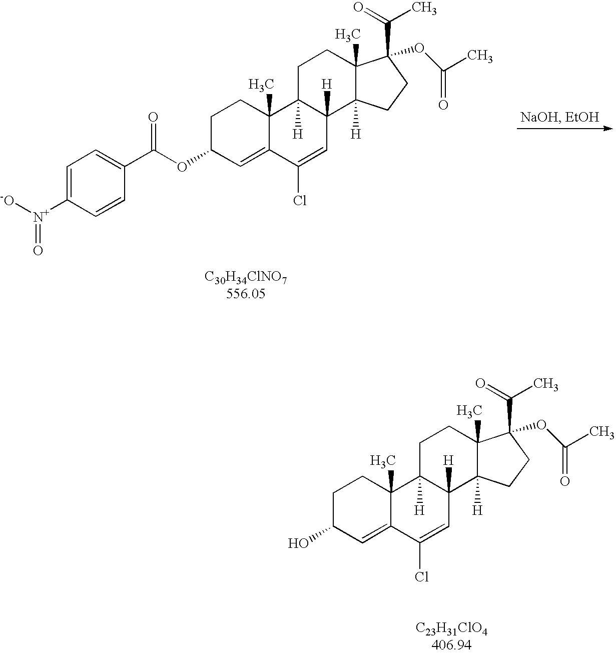

3α-(4-nitrophenylcarboxy)chlormadinone acetate (338 mg; 0.61 mmol) was placed in ethanol (12.2 ml), to which 6 N sodium hydroxide solution (105 μl; 0.63 mmol) was added, and the mixture was stirred for 45 minutes. The pH was brought to between 7.0 and 7.5 by using 5% hydrochloric acid and the mixture was evaporated. The residue was laced with water and extracted three times using dichloromethane. The combined organic phases were dried over sodium sulphate, filtered off from the desiccant and evaporated. The residue was purified by flash chromatography (silica gel; chloroform/dichloromethane/diethyl ether: 5/5/1).

| | |
|---|---|
| Yield: | 171 mg (69.0%), colourless solid |
| Melting point: | 134.6° C. |
| α/β ratio: | 95.1/4.9 (¹H-NMR) |
| | 95.6/4.4 (HPLC) |
| Amount of rotation: | [α]_{D} = +39.2 (c = 1.237; CHCl₃) |
| LC-MS: | m/z = 406.9 (MH⁺) |

¹H-NMR (400 MHz, CDCl₃): δ=0.70 (s, 3H); 0.95 (s, 3H); 1.12-2.02 (m, 14H); 2.05 (s, 3H); 2.09 (s, 3H); 2.26 (t, J=12.6 Hz, 1H); 2.97 (t, J=10.5 Hz, 1H); 4.32 (s, 1H); 5.94 (s, 1H); 6.22 (s, 1H) ppm.

¹³C-NMR (100 MHz, CDCl₃): δ=14.33; 16.74; 20.53; 21.18; 23.25; 26.40; 27.02; 29.57; 30.26; 31.16; 37.07; 38.10; 47.45; 49.14; 50.19; 63.15; 96.39; 124.83; 130.09; 131.03; 142.91; 170.67; 203.96 ppm.

### EXAMPLE 2

The effect of the reaction process on the diastereomer ratio in the Mitsunobu reaction was investigated.

### General Reaction Specifications:

3β-hydroxychlormadinone acetate (1.50 mmol; 61 mg) and triphenylphosphine (1.62 mmol; 426 mg) were placed in 11 ml absolute solvent under a nitrogen atmosphere and stirred for 10 minutes. 4-nitrobenzoic acid (1.72 mmol; 288 mg) was subsequently added and the mixture was stirred for a further 15 minutes. Diethyl azodicarboxylate (1.57 mmol; 0.74 ml in 2.2 ml absolute solvent) was diluted with 11 ml solvent and was added slowly at temperature T₁. The batch was stirred at temperature T₂ for 18 hours. It was then evaporated and the residue was added to dichloromethane (25 ml) and washed with sodium carbonate solution and water. After it was dried over sodium sulphate, it was filtered off from the desiccant and evaporated. The residue was purified by flash chromatography (silica gel; chloroform/dichloromethane/diethyl ether: 5/5/1).

| | (a) | (b) | (c) | (d) | (e) |
|---|---|---|---|---|---|
| Solvent | THF | THF | CH₂Cl₂ | Toluene | Benzene |
| T₁ | 21° C. | 0-5° C. | 21° C. | 21° C. | 21° C. |
| T₂ | 21° C. | 5-8° C. | 21° C. | 21° C. | 21° C. |
| α/β ratio | 81.8/18.2 | 79.2/20.8 | 76.1/23.9 | 95.7/4.3 | 96.5/3.5 |

As the data in the above table show, lowering the reaction temperature results in deterioration in the diastereomer ratio [(a) vs. (b)]. Furthermore, markedly improved diastereomer ratios were achieved in non-polar aromatic solvents [(d) and (e)] than in comparatively polar non-aromatic solvents [(a), (b) and (c)].

1. A process for preparing 3α-hydroxychlormadinone acetate (I): 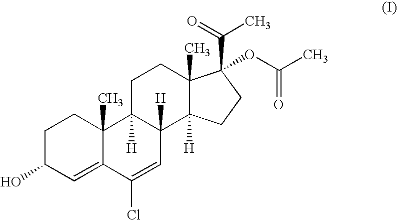
said process comprising:
a) reducing chlormadinone acetate (II): 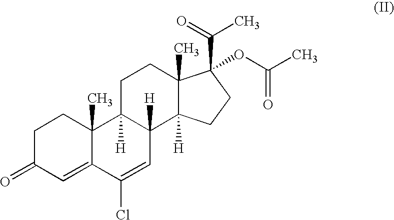
with a hydride component to form 3β-hydroxychlormadinone acetate (III): 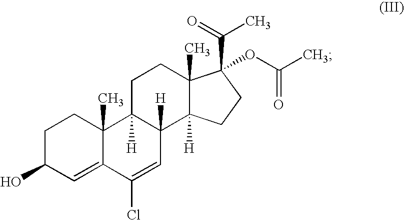
b) inverting the configuration of 3β-hydroxychlormadinone acetate (III) in position 3 to form 3α-hydroxychlormadinone acetate (I).

2. Process according to claim 1, wherein
the substance amount ratio (mol/mol) of the hydride component to chlormadinone acetate (II) in step a) is at least 1.38, and/or
the chlormadinone acetate (II) concentration in step a) is between 0.0010 and 1.0 mol/l, and/or
the concentration of the hydride component in step a) is between 0.010 and 10.0 mol/l.

3. Process according to either claim 1, wherein the hydride in step a) is achiral.

4. Process according to claim 1, wherein the hydride is a metal hydride comprising at least one metal selected from the group consisting of lithium, sodium, potassium, magnesium, calcium, boron, aluminium, silicon, tin and zinc.

5. Process according to claim 1, wherein said inverting comprises:
b1) reacting 3β-hydroxychlormadinone acetate (III) with at least one carboxylic acid in the presence of:
b1′) at least one phosphine component and at least one azo component, or
b1″) at least one phosphorane component
to form an ester of the formula (IV): 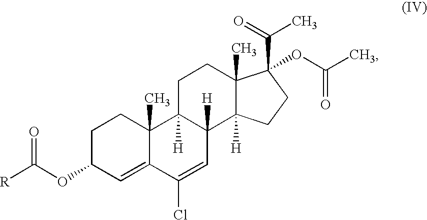
in which R is an unsubstituted or a mono- or polysubstituted C₁-C₁₂ hydrocarbon; and
b2) cleaving the ester (IV) to form 3α-hydroxychlormadinone acetate (I).

6. Process according to claim 5, wherein the carboxylic acid has the formula R—COOH, in which R is an unsubstituted or a mono- or polysubstituted C₁-C₁₂ hydrocarbon.

7. Process according to claim 5, wherein the phosphine component in step b1′) is a compound of formula (V): 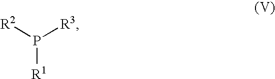
in which
R¹, R² and R³, are each selected, independently of one another, from the group consisting of C₁-C₁₂ alkyl, C₃-C₁₂ cycloalkyl, aryl and heteroaryl, each of which are unsubstituted or mono- or polysubstituted with the same or different residues selected from the group consisting of halogen, —CN, —NO, —NO₂, —C₁-C₆ alkyl, —C₁-C₆-perhalogenalkyl, —O—C₁-C₆ alkyl, —S—C₁-C₆ alkyl, —N(C₁-C₆ alkyl)₂, pyrrolidinyl, piperidinyl, N-methylpiperazinyl, morpholinyl, —NHC(═O)—C₁-C₆ alkyl, —NH(C═O)O—C₁-C₆ alkyl, —C(═O)—C₁-C₆ alkyl, —C(═O)O—C₁-C₆ alkyl and —C(═O)NH—C₁-C₆ alkyl.

8. Process according to claim 5, wherein the azo component in step b1′) is a compound of formula (VI): 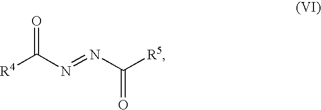
in which
R⁴ and R⁵ are each selected, independently of one another, from the group consisting of C₁-C₁₂ alkyl, —N(C₁-C₆ alkyl), pyrrolidinyl, piperidinyl, N-methylpiperazinyl, morpholinyl and aryl, each of which are unsubstituted or mono- or polysubstituted with the same or different residues selected from the group consisting of halogen, —CN, —NO₂, —C₁-C₆ Alkyl, —C₁-C₆ perhalogenalkyl, —O—C₁-C₆ alkyl, —S—C₁-C₆ alkyl and —N(C₁-C₆ alkyl)₂; or
R₄ and R₅ together form the group 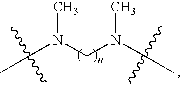
in which n represents 1, 2, 3 or 4.

9. Process according to claim 5, wherein the phosphorane component in step b1″) is a compound of formula (VII): 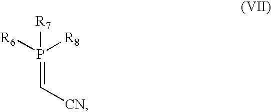
in which
R⁶, R⁷ and R⁸ each represent, independently of one another, a C₁-C₁₂hydrocarbon.

10. Process according to claim 5, wherein the ester is cleaved by means of acids or bases.

11. A compound of formula (IV): 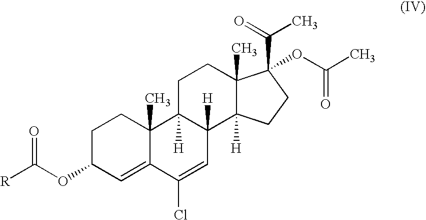
wherein R is an unsubstituted or a mono- or polysubstituted C₁-C₁₂ hydrocarbon, provided that R is not ethyl.
